(19) United States
(12) Patent Application Publication
(10) Pub. No.: US 2016/0060677 A1
(43) Pub. Date: Mar. 3, 2016

(54) DEVICE FOR THE MICROBIOLOGICAL DETERMINATION AND ANALYSIS OF A PREPARED LIQUID SAMPLE

(71) Applicant: D. Roy Cullimore, Regina (CA)

(72) Inventor: D. Roy Cullimore, Regina (CA)

(21) Appl. No.: 14/470,421

(22) Filed: Aug. 27, 2014

(51) Int. Cl. C12Q 1/04

(52) U.S. Cl. CPC C12Q 1/04

(57) ABSTRACT

The device comprises a microbiologically interactive platform within a liquid-based environment. The platform is positioned by a vertical positioning pin rising centrally from the floor of the device with a floating ball within the device that angles the platform in a specific manner that creates a multiplicity of environments within the device. While the device remains dry, there are no significant microbiological interactions. When a suitably prepared liquid sample is added then there are interactions that allow the determinative analysis of any generated microbiologically generated reactions and responses. These activities are generated primarily from the positioning of the microbiologically interactive platform wherein the platform is formed into an angled profile positioned by the floating ball and the base of the vertical extension pillar. When so positioned, both oxidative and reductive environments are created. Oxidative conditions dominate the platform on the upper side which is directly under the floating ball and where the extension remains exposed. Reductive conditions dominate the lower side of the platform.

### BACKGROUND OF THE INVENTION

The present invention relates to the microbiological analysis of a liquid sample which may contain any combination planktonic, suspended and /or porous or fractured materials considered to contain viable particles of interest.

The preparation of the liquid sample which may contain viable microorganisms involves the use of the device which is specifically designed to detect communities of bacteria within the sample being investigated and evaluate their activities.

Such preparations of the sample are known from the U.S. Pat. No. 5,187,072 through the application of the liquid sample into the device. U.S. Pat. No. D687,512 is employed to modify the surface area of the floating ball with a series of ribs that increase the surface area of the ball and provided cavities within which microbes then attach and grow in visibly differentiable manners.

In the development of this invention regarding the ribbed floating ball, it became recognized that further improvements could be made to the device disclosed in U.S. Pat. No. 5,187,072 and U.S. Pat. No. D687,512. It became evident that a number of unique features could be incorporated into the device. This invention relates to accelerating the biological activities and shortening the reaction times in the investigative process.

### BRIEF SUMMARY OF THE INVENTION

According to this invention, there is a porous membrane that is positioned totally within the sample confined within the device. This membrane contains a designed selective chemistry that allows interaction with the microorganisms within the sample that then have determinative diagnostic value. This component forms a unique microbiologically interactive platform.

To ensure the correct positioning of the microbiologically interactive platform there is a centrally located vertical extension which rises up from a central location in the cone-shaped floor of the device.

These two additions to the device allow the precise positioning of the microbiologically interactive platform angled between the foot of the vertical extension at the base and the floating ribbed ball. This assures a consistent angle for the platform within the device. Such precise positioning allows the development of a number of microbiologically influenced activities that have potential analytical value in the diagnosis of the water sample.

The device, according to this invention, by means of the microbiologically interactive platform, causes the radical diffusion of the implanted chemicals and other adjuvants upon the addition of the liquid sample to the device. These releases are controlled by the rapid admission of the liquid sample to the platform by diffusion and solubilisation of the entrenched chemicals within the dried void spaces. For the previous apparati, such chemicals were presented as a crystallized cone and releases were subjected to the delays associated with the limited dissolution and diffusion fronts formed along the interface between the liquid sample and the crystallised pellet in the floor. The microbiologically interactive platform provides more radical dissolution and diffusion in and out from the platform. These phenomena lead to an accelerated rate of natural movement from the platform into the liquid sample contents.

Additionally the employment of a vertical centralized positioning pin stabilizes positioning of the sloping microbiologically interactive platform. Here the base of the platform rests against the base of the positioning pin while the top is contained between the floating ball and the inner side wall of the vial. This entrapment gives the platform a gentle slope outwards from the base of the vertical positioning pin between the floating ball and the inner wall of the apparatus. This engineered slope of the platform from under the ball at the top to the centralized pillar at the bottom creates a confinement region with unique environmental characteristics. The underside of the platform has a more restricted access to diffusing oxygen from the headspace above the liquid sample and the floating ball. Such a region has more limited access to diffusing oxygen primarily from the headspace of the device on the underside of the platform. This environment is more oxygen stressed and more reductive than the liquid environment outside of the platform. The uppermost side of the platform, totally immersed within the liquid culturing medium, has direct access to any free dissolved oxygen penetrating downwards into this part of the liquid medium. This more direct access to diffusing oxygen causes the upper side of the platform to transition to oxidative environments while the lower side transitions towards more reductive states.

One set of unique features of the microbiologically interactive platform is that its porous nature allows the implantation of various selected chemical and/or biological agents into the voids while the platform is being prepared. These features release agents from the platform as well as receive agents onto and into the matrices of the platform. Within this interaction between agents entering into the platform and those agents present in or leaving the platform, definable events have analytical value in defining specific factors within the liquid sample in the device.

Preferred features for the device relate to the use of a porous microbiologically interactive platform with the liquid sample under investigation for analytical purposes and to the vertical positioning pin within the device set to limit the positioning of the platform within the device.

### BRIEF DESCRIPTION OF THE SEVERAL VIEWS OF THE DRAWINGS The features and advantages of the invention will appear from the following description with reference to the accompanying drawings in which:

FIG. 1 is an elevational view showing the essential features of the device according to this invention;

FIG. 2 is an elevational view showing movement of elements within the device;

FIG. 3 is an elevational view rotated 90° from that shown in FIG. 2;

FIGS. 4, 5 and 6 are elevational views showing the internal elements of the device in various orientations; and

FIG. 7 is a bottom plan view.

### DETAILED DESCRIPTION OF THE INVENTION

This device is designed to be used once by the addition of the liquid sample of interest. Such a sample may be clear, cloudy or formed as a suspension of solid or porous matrix that is suspected to contain viable organisms. For clear liquid samples, these are directly dispensed into the device. For liquid samples that are clouded by suspended material with visibility obscured, a tenfold or one hundred fold dilution in a suitable sterile medium would become necessary. For porous or solid samples, there is a need to employ dilution. Common practice dictates a one in ten or one in one hundred dilution in a suitable sterile liquid medium. In these events, the best practice is to remove the microbiologically interactive platform and the ball aseptically and then add the defined acceptable weight of the solid or porous sample followed by the return of the platform and the ball prior to addition sterile liquid to optimize the functionality of the device.

In practice, the device is subjected to a period of incubation commonly at a desired temperature that then triggers signals and events that might be considered significant in the analytical process. The basic elements of the device include generally circular container 101 with inverted cone-shaped bottom 102. Threaded top 103 is utilized to effectively close container 101.

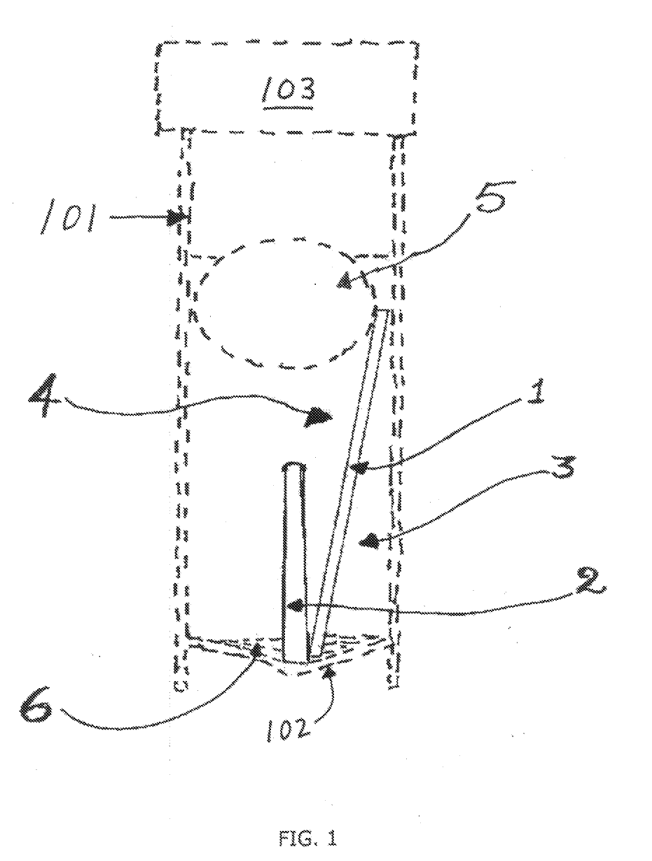

The analytical procedures, according to this invention, relate to the form deployment of two critical key physical components within the device. These are illustrated in FIG. 1 as platform 1 and positioning pin 2. Here vertical positioning pin 2 significantly limits the movement of microbiologically interactive platform 1. Platform 1 is held in position by floating ball 5 that also separates the liquid environment into oxidative zone 4 and reductive zone 3 where differences are likely to occur. Additional chemical and/or biological agents may be deployed in base 6 to further trigger biologically influenced analytical activities.

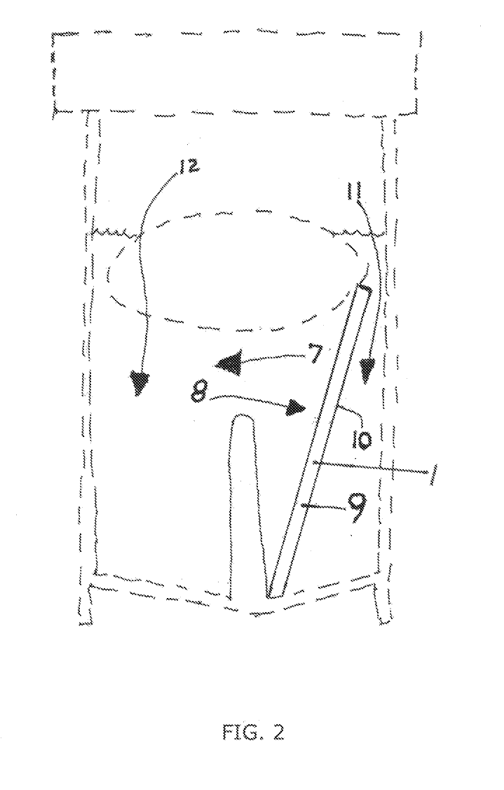

Critical are the analytical roles that are affected by platform 1 and illustrated in FIG. 2. Here platform 1 is sloped and held within the device that causes the ingress 8 and egress 9 of agents moving into and out of platform 1 once the device is charged with a suitable liquid sample. Concurrently there is movement of such agents around platform 1 leading to confinement within the voids 9 and on surfaces 10. Oxygen from the headspaces gases originating from the atmosphere will diffuse into the sample retained within the device in a restricted manner above pathway 11 and more effectively around pathway 12 thus aiding in creating both oxidative and reductive zonation within the device.

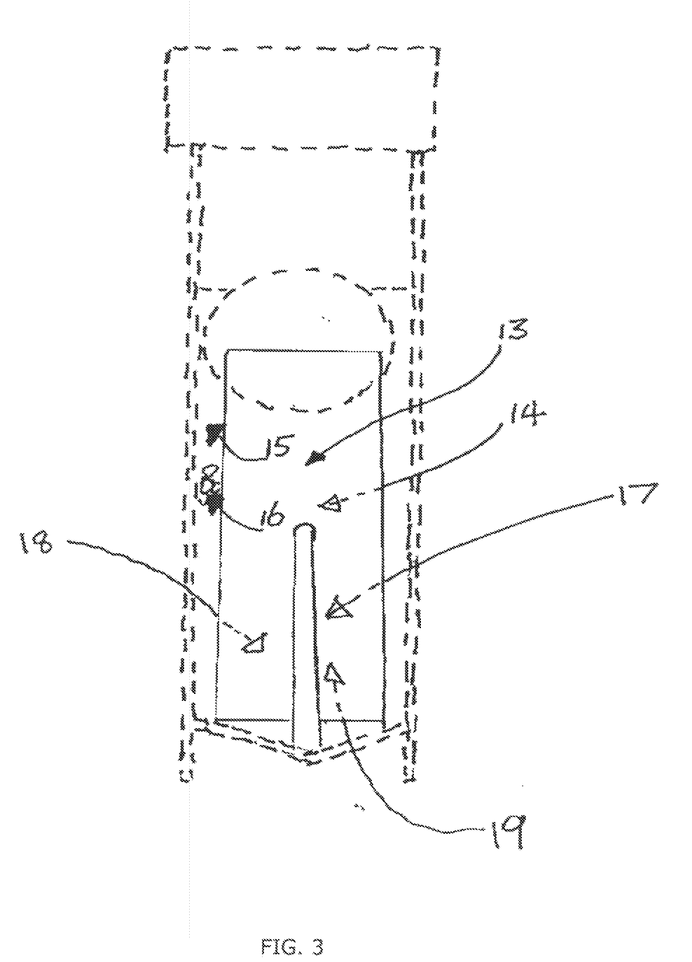

The interactions potentially possible between platform 1 and the living organisms that are present and active within the liquid sample are shown in FIG. 3. Here platform 1 provides a multiplicity of activities with the surrounding microflora. Microorganisms are present in the samples in a number of mechanisms and entrenched at 13 or attached as biofilms 14 but can be aerobic 15, biocolloids 16 or anaerobic 17, 18 and 19 which means that a complex series of interactions are expected to occur between the platform and the surrounding liquid sample that also saturates the voids.

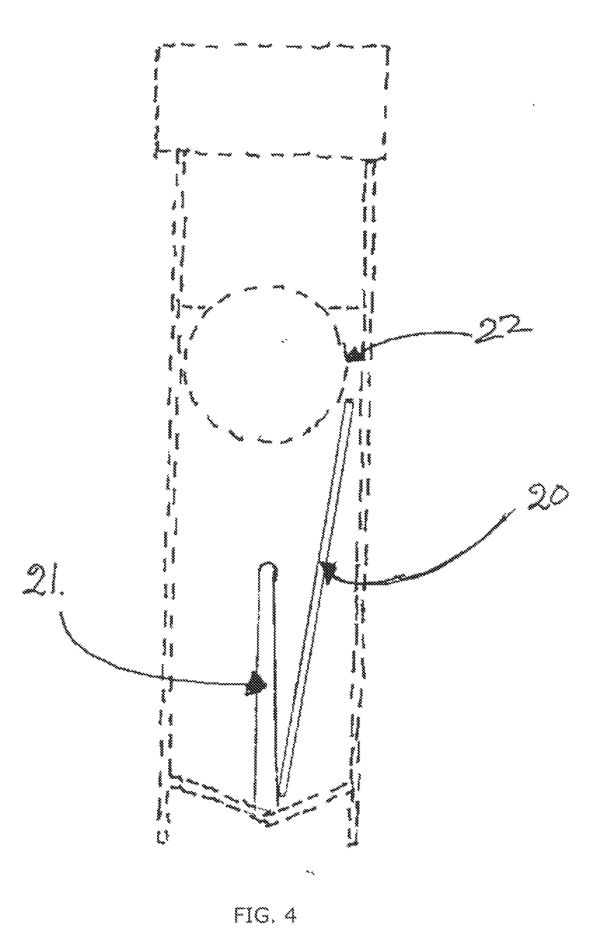

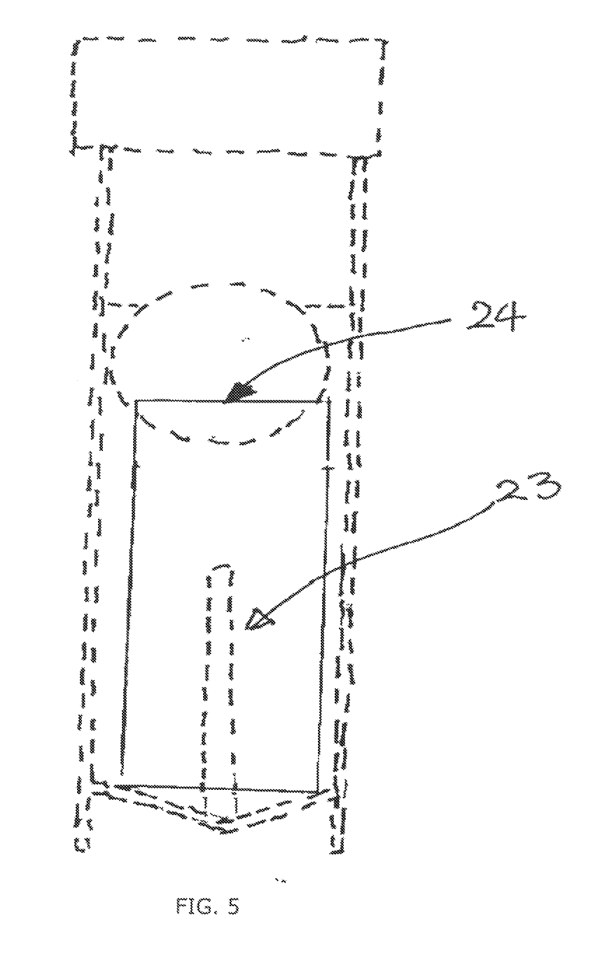

Positions of the microbiologically interactive platform and the vertical positioning pin rising from the floor of the device are illustrated in FIGS. 4 to 7. A side view of the device is shown in FIG. 4 illustrating the relative positions platform 20 is pivoted and restricted by pin 21. There is an automatic confinement of the platform at the top by the wall of the floating ball. When viewed from the rear perspective in FIG. 5, the position of the platform is held in a vertical but sloping position by edges 22 of the floating ball. The top of the platform restricts access to any diffusing oxygen moving downwards from the headspace gases. The net effect of this confinement is a reduced rate of oxygen diffusion which could then trigger reductive conditions 24 with very little penetration on that environment with diffusing oxygen.

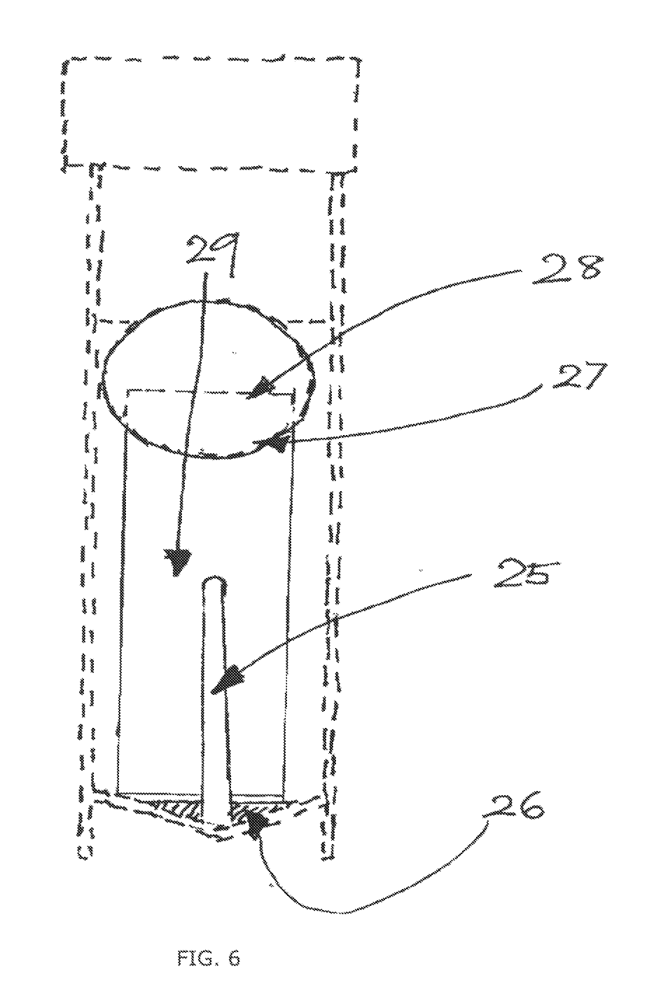

When viewed from the front of the device in FIG. 6, the vertical positioning pin is fully visible at 25 except at the very bottom of the device and only in designed conditions deposits 26 are concentrated on the inner conical floor of the device. Here platform 27 becomes invisible behind floating ball 28 and extension 25. Headspace oxygen diffuses more effectively around floating ball 28 to render more oxidative conditions on this side of platform 29.

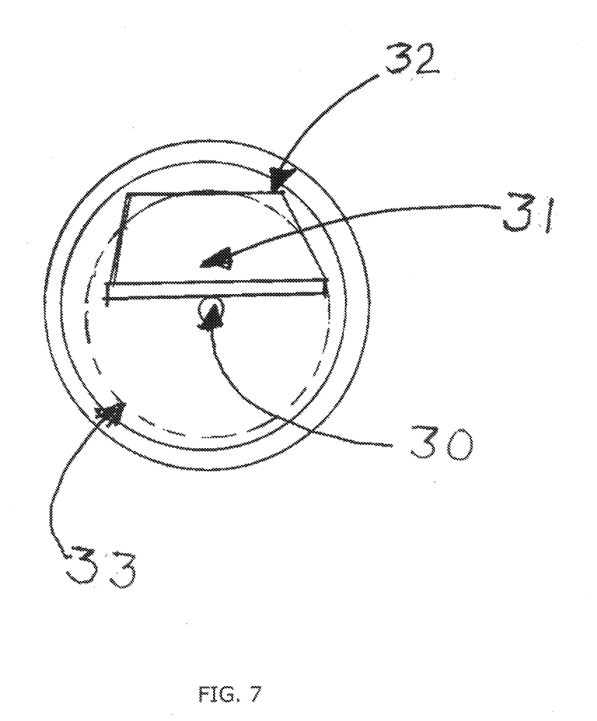

In FIG. 7, the view is from below through the device. Here the positioning pin is positioned centrally to allow the platform to settle upon extension 30 at the base of extension 31 while the top of the platform is confined by the floating ball which then causes a gap 32 to be created so that diffusate oxygen penetrates into the more reductive side of the platform. Gapping 33 shifts the floating ball to a locked location in which the ball becomes stabilized.

### NUMERAL GLOSSARY

FIG. 1
1. Microbiologically interactive platform
2. Vertical positioning pin from the floor of the device
3. Oxygen restricted (reductive) zone
4. Oxygen unrestricted (oxidative) zone
5. Water to headspace air interface around the floating ribbed ball
6. Basal site for the possible addition of additional chemicals to the device

FIG. 2
7. Diffusion pathway for agents applied to the platform into the liquid sample
8. Diffusion pathway for agents in the liquid sample to enter onto, or into, the platform
9. Void volumes within the platform that would become saturated with the liquid sample when applied to the device
10. Textured surface area on the platform that may causate discernable events as a result of the application of the liquid sample to the device
11. Pathway for potential oxygen diffusion at the floating ball and the leaning platform interface with the wall of the device when liquid sample is applied
12. General oxygen pathway into the liquid sample contained within the device and outside of the platform

FIG. 3
13. Attachment of agents from the liquid sample on the surface of the platform such as biofilms
14. Entrenchment of biological agents to within the void spaces of the platform
15. Interaction between the agents applied to the platform and planktonic agents suspended within the liquid sample outside of said platform
16. Interaction between the agents applied to the platform and particulated viable agents suspended within the liquid sample
17. Zone on the platform which would have limited access to diffusing oxygen and would encourage the planktonic viable agents to become active
18. Zone on the platform which would have restricted access to diffusing oxygen and would encourage the suspended particulates carrying viable agents to become active
19. Zone on the platform where there would be a greater probability interaction between the surfaces of the platform and anaerobic viable agents

FIG. 4
20. Side view of microbiologically interactive platform set in sample charged device
21. Vertical positioning pin limiting the platform at the base while the floating ball restricts the platform at the top
22. Floating ball pushed towards the oxidative side by the platform coming into contact with the ball

FIG. 5
23. View of the platform from the reductive side of the device
24. Oxygen entry into the reductive side of said platform is restricted to a small gap primarily between said platform and the curved inner wall of the device

FIG. 6
25. Vertical position of the platform viewed from the front
26. Optional position for additional agents to be applied to the device if required to complete the analysis
27. View from front showing the floating ball in front of the platform into a stabilized position
28. The platform held in a stable manner behind the floating ball once the liquid sample has been added
29. Oxygen from the headspace diffuses through the air: liquid interface to create oxidative conditions

FIG. 7
30. Position of the vertical positioning pin looking directly upwards through the device
31. View of the platform when viewed from beneath the device with the reductive side uppermost
32. Interface between the top of the platform and the inner wall of the device showing a restricted area for oxygen to penetrate the platform on the (uppermost) reductive side
33. Position of the floating ball in the device filled with the liquid sample viewed from beneath.

1. A device for the microbiological determination analysis of a liquid sample comprising a closed container, said container having a bottom, a positioning pin extending upwardly from said bottom, and a microbiological platform detachably disposed in said container.

2. The device according to claim 1 wherein said container is circular.

3. The device according to claim 2 wherein liquid is disposed in said container and a ball is disposed in said liquid.

4. The device according to claim 3 wherein said platform is angularly disposed with respect to the axis of said container.

5. The device according to claim 4 wherein said platform comprises a lower portion and said lower portion is in abutting relation with said pin.

6. The device according to claim 5 wherein said platform comprises an upper portion and said upper portion is in abutting relation with said ball.

7. The device according to claim 6 wherein said ball is disposed laterally on one side of said platform.

8. The device according to claim 1 wherein liquid is disposed in said container and said liquid disposed on one side of said platform is oxidative.

9. The device according to claim 8 wherein the liquid disposed on the opposite side of said platform is reductive.
